(19) United States
(12) Patent Application Publication
(10) Pub. No.: US 2010/0222687 A1
(43) Pub. Date: Sep. 2, 2010

(54) METHOD AND SYSTEM FOR MONITORING VITAL BODY SIGNS OF A SEATED PERSON

(75) Inventors: Jeroen Adrianus Johannes Thijs, Eindhoven (NL); Jens Muhlsteff, Aachen (DE); Robert Pinter, Aachen (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/679,316

(22) PCT Filed: Sep. 19, 2008
(86) PCT No.: PCT/IB2008/053812

§ 371 Date: Mar. 22, 2010

(30) Foreign Application Priority Data
Sep. 25, 2007 (EP) 07117151.6

(51) Int. Cl. A61B 5/02

(52) U.S. Cl. 600/508

(57) ABSTRACT

A method comprising the step of using a plurality of doppler radars disposed on the seat belt or integrated into the seat belt for monitoring vital body signs of a person seated in a seat of a motor vehicle is disclosed. The disclosed method unobtrusively monitors vital body signs like heart rate and respiration of the person seated in the motor vehicle. A number of safety applications as well as wellness applications can be enabled. Examples are detection of momentary sleep of the driver, vital sign monitoring in case of an accident as well as relaxation exercise using biofeedback to reduce stress for drivers.

### FIELD OF THE INVENTION

The present subject matter relates to a method and system for monitoring vital body signs of a seated person, and specifically for monitoring vital body signs of a person seated in a motor vehicle.

### BACKGROUND OF THE INVENTION

Patent document US2005/0073424 discloses a method for sensing information about the position and/or movements of the body of a living being in particular for use in a motor vehicle. The method uses doppler radar sensor integrated in the steering wheel of the car to enable monitoring vital body signs of the driver from a distance. The monitoring of vital body signs of the driver may not be accurate because other moving objects around the driver can cause signal artifacts' in the doppler radar signal.

It would be advantageous to have a method that can improve the accuracy of monitoring vital body signs of a person seated in a motor vehicle.

It would also be advantageous to have a system that can improve the accuracy of monitoring vital signs of a person seated in a motor vehicle.

### SUMMARY OF THE INVENTION

A method comprising the step of using a plurality of doppler radars disposed on the seat belt or integrated into the seat belt for monitoring vital body signs of a person seated in a seat of a motor vehicle is disclosed.

A system for monitoring vital body signs of a person seated in a seat of a motor vehicle is disclosed. The system comprises a plurality of transducers and antennas to transmit electromagnetic signals of a certain frequency into the chest of the person and receive corresponding reflected electromagnetic signals from the chest of the person. The system comprises a processing unit. The processing unit comprises a first processing unit, coupled to the plurality of antennas to process the reflected electromagnetic signals and produce output signals, the output signals representing the rate of change of the doppler signal associated with the reflected signal, the rate of change with respect to time. The processing unit comprises a second processing unit, arranged to compare the output signals and select the best output signal based on a criteria. The processing unit comprises a third processing unit, arranged to calculate at least one parameter representative of the vital body sign of the person seated in the seat of the motor vehicle based on the selected best output signal.

### BRIEF DESCRIPTION OF THE DRAWINGS

The above-mentioned aspects, features and advantages will be further described, by way of example only, with reference to the accompanying drawings, in which the same reference numerals indicate identical or similar parts, and in which:

FIG. 1 shows an exemplary arrangement for monitoring vital body signs of a person seated in a motor vehicle;

FIG. 2 schematically shows the steps involved in monitoring the vital body signs of the person seated in a motor vehicle according to an embodiment of the subject matter;

FIG. 3 shows a flowchart and a graph illustrating the selection of the best output signal according to an embodiment of the subject matter; and

FIG. 4 shows an embodiment of the system for monitoring vital body signs of a person seated in a motor vehicle.

There is a great deal of interest in the automotive industry regarding the safety of the vehicle operator because inattentiveness, falling asleep at the wheel and cardiac stress caused by stressful situations are frequent causes of accidents with fatalities.

The present subject matter discloses an improved method and system for monitoring vital body signs of the vehicle operator.

The word vehicle here refers to conveyance that transports people or objects (e.g. car, bus, truck, ambulance). The word vehicle operator here refers to a person who drives/operates the vehicle.

### DETAILED DESCRIPTION OF THE EMBODIMENTS

A method comprising the step of using a plurality of doppler radars disposed on the seat belt or integrated into the seat belt for monitoring vital body signs of a person seated in a seat of a motor vehicle is disclosed.

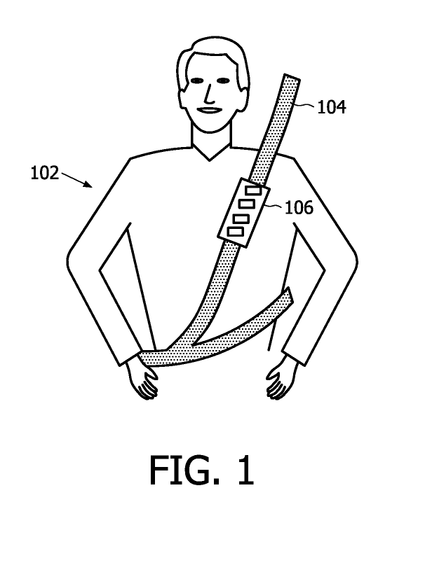

Referring now to FIG. 1, a person 102 is seated in a seat of a motor vehicle wearing a seat belt 104. The seat belt 104 here refers to a safety belt designed to secure the person 102 against harmful movement that may result from a collision or a sudden stop. The seat belt 104 is intended to reduce injuries by stopping the person 102 from hitting hard interior elements of the vehicle or other passengers and by preventing the person 102 from being thrown from the vehicle. A plurality of doppler radar's 106 are disposed on the seat belt or integrated into the seat belt. The doppler radars are used to measure vital body signs of the person 102.

Transducers for the detection of doppler shifted signals are commercially available, and are often used for the purposes of detection of movement using the far field of the beam, for example in Radar measurements of traffic speed. Such transducers can also be used for near field measurements and are suitable for detecting heart activity via the detection of doppler shifted signals from the heart.

One such commercially available transducer is Microwave Motion Sensor KMY 24 unit, a two channel motion sensor, made by Micro Systems Engineering GmbH. It contains a 2.45 GHz oscillator and receiver in the same housing and works in continuous wave mode.

Generally in such doppler transducers, as is known in the art, an antenna emits an electromagnetic wave which, when it is reflected from the surfaces of an object moving with a component of velocity non-transverse to the impinging electromagnetic wave, produces a shift in the frequency of the electromagnetic wave reflected back to the antenna. This shift in frequency is called the doppler shift. This doppler shifted reflected wave is detected by an antenna in the transducer, which may or may not be the same antenna as the emitting antenna. The relative speed of movement of the reflecting object is encoded in the frequency shift of the detected reflected electromagnetic wave and this value can be extracted using known techniques.

The solution disclosed in US 2005/0073424 uses a single sensor integrated in the steering wheel. Measuring the vital body signs using doppler radars disposed on the seat belt 104 gives a much better signal activity and much less susceptenance to moving objects around the driver than when integrating into the steering wheel.

Because an exact positioning of the doppler radar above the heart region of the seated person cannot be guaranteed, a doppler radar array consisting of multiple doppler radars is used. Multiple radars are arranged next to each other on or integrated into the seat belt. Their respective data output and power supply leads are integrated as shielded conductive wires in the seat belt.

Multiple radars present an advantage in the exact positioning of the doppler radar sensors, making it insensitive to the position of the driver and the setting of the driver's seat (e.g. angle of the back seat). Multiple signals can be obtained and the most useful signal can be selected thereby enabling measurement of the vital body signs with higher accuracy. The vital body signs such as heart rate and respiration can be monitored without skin contact and are completely unobtrusive to the driver.

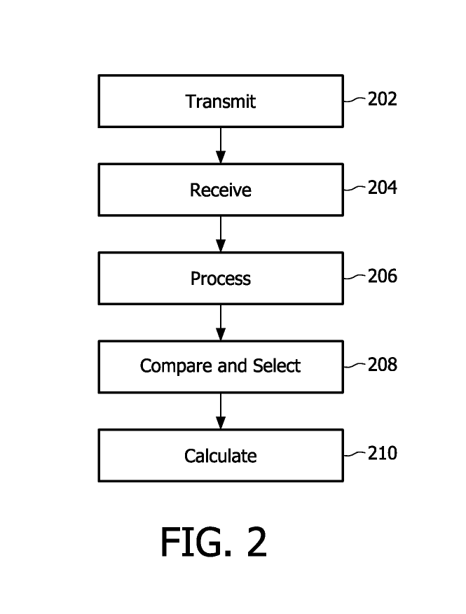

In an embodiment, monitoring the vital body signs of the seated person 102 comprises the following steps as shown in FIG. 2. Step 202 involves transmitting electromagnetic signals of a certain frequency into the chest of the person seated in the seat of the motor vehicle. Step 204 involves receiving corresponding reflected electromagnetic signals from the chest of the person seated in the motor vehicle. Step 206 involves processing the corresponding reflected electromagnetic signals to produce output signals representing the rate of change of the doppler signal associated with the reflected electromagnetic signal, the rate of change with respect to time. Step 208 involves comparing the output signals and selecting the best output signal based on criteria. Step 210 involves calculating at least one parameter representative of the vital body sign of the person based on the selected best output signal. The disclosed method does not measure the impedance, but the chest wall and the heart wall movement.

In a further embodiment, comparing the output signals and selecting the best output signal comprises selecting the best output signal based on heart signal of the person seated in the motor vehicle. The best output signal is selected based on the number of characteristic points the signal shows in one cycle. In case of small displacements of the sensor due to breathing or other movements, the sensor which had the best signal is very likely to remain the sensor with the best signal after the small movement, since it will still be the closest to the heart. It is therefore advantageous to not just take any sensor that outputs a repeating pattern, but take the one with the most characteristic points per cycle.

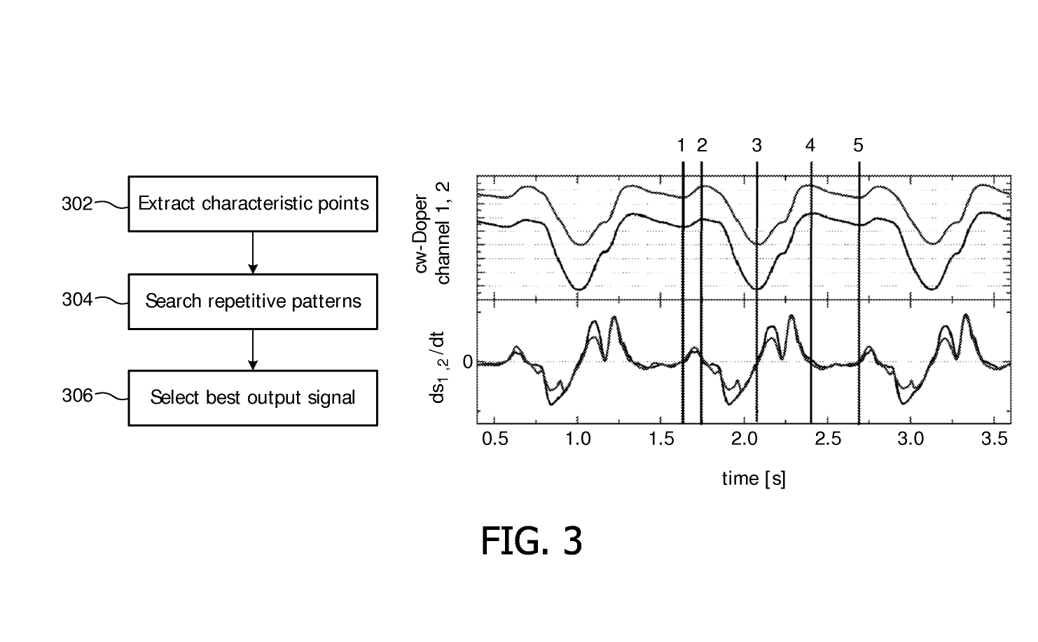

In the European patent application PHNL 006855, the use of two channel doppler radar sensor for heart measurements is described that provides information about timing of heart phases. This has been further described in the paper titled “The use of a two channel doppler radar sensor for the characterization of heart motion phases” by J. Muehlsteff, J. A. J. Thijs, and R. Pinter, 28^{th} Annual International Conference of the IEEE, Engineering in Medicine and Biology Society 2006, EMBS 06, pages 547-550. In the results presented in this paper, there are four characteristic points in one RR cycle (Cf FIG. 3 and note that point 5=point 1). The inventors have found out that depending on the position on the thorax not all the four characteristic points are visible all the time. If the four characteristic points are visible in the output signal, this implies the measurement position is a good one. This can be used to select the right sensor in the seat belt which would then be the sensor that has the most characteristic and plausible points in one RR cycle.

The characteristic points and the time differences between these subsequent characteristic points are calculated from the reflected signals. This can give a repeating pattern up to four characteristic points which keep repeating with the heart frequency. This enables to find out the most advantageously positioned sensor. This can be done by calculating how many characteristic points per RR cycle are visible. Selecting the best output signal based on the heart signal comprises the following steps as shown in FIG. 3. Step 302 involves extracting characteristic points from all the radar sensors using the time derivatives of all the radar channels. Step 304 involves searching for repeating patterns of characteristic points. Step 306 involves selecting best output signal based on the number of characteristic points in one repeating pattern (i.e. RR cycle) which is graphically depicted in FIG. 3

In a still further embodiment, the pluralities of doppler radars emit continuous wave electromagnetic signals at a frequency in a range between 400 MHz and 5 GHz. This range is found to be particularly advantageous for producing signals which are reflected from the heart. However, the method works in a particularly advantageous manner when the frequency is in a range of between 800 MHz and 4 GHz.

In a still further embodiment, the monitored information about the vital body signs of the person are forwarded to a higher-order system for further processing for at least one of the following purposes:

detecting momentary sleep of the person seated

classifying the health condition of the person seated

giving feedback on the health condition of the person seated.

The health condition of the person can be continuously monitored and the feedback can help the person in being attentive thereby reducing accident.

In a still further embodiment, the method comprises generating an alarm signal when the monitored information about the vital body signs of the person seated indicates a life-threatening or abnormal situation. By alerting the driver, accident can be avoided.

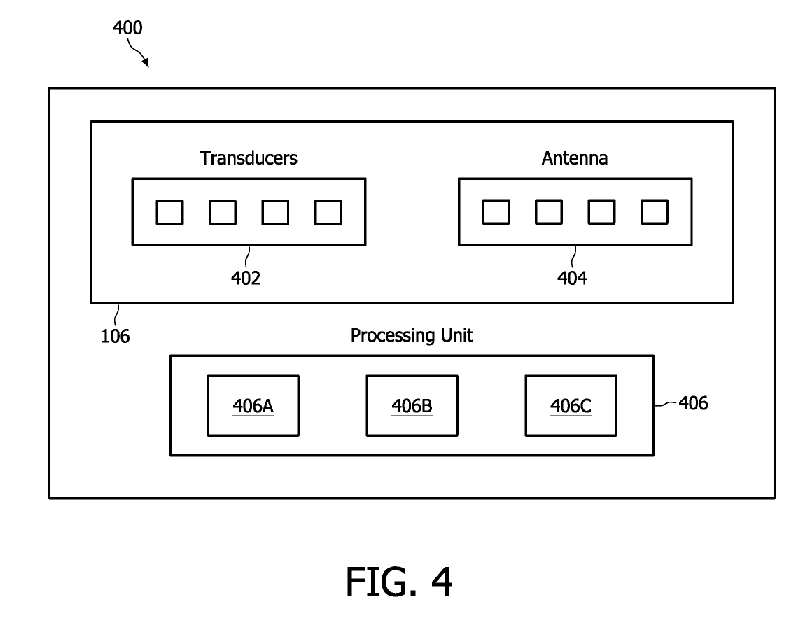

Referring now to FIG. 4, a system for monitoring the vital body signs of a person seated in the seat of a motor vehicle comprises:

a plurality of doppler radars 106 comprising a plurality of transducers 402 and a plurality of antennas 404

a processing unit 406 comprising
a first processing unit 406A
a second processing unit 406B and
a third processing unit 406C.
   The pluralities of transducers and antennas can be mounted on the seat belt. There may be wires that connect the transducers and antennas to the processing unit 406. The wires can be integrated into the seat belt as shielded conductive yarns. Wireless solutions are also possible. However, in this case, the sensors have to be battery powered and regularly recharged. Further, the processing unit 406 can be housed anywhere in the motor vehicle.

The first processing unit 406A is coupled to the plurality of antennas to process the reflected electromagnetic signals and produce output signals, the output signals representing the rate of change of the doppler signal associated with the reflected signal, the rate of change with respect to time.

The second processing unit 406B is arranged to compare the output signals and select the best output signal based on a criteria and the third processing unit 406C is arranged to calculate at least one parameter representative of the vital body sign of the person seated in the seat of the motor vehicle based on the selected best output signal.

The processing unit 406 makes use of the methods disclosed in the embodiments to process the reflected electromagnetic signals and select the best output signal.

The disclosed method is unobtrusive and comfortable for monitoring vital body signs like heart rate and respiration in a motor vehicle such as car, bus, truck and ambulance. Safety applications include but not limited to detection of momentary sleep of the driver, vital body sign monitoring in case of an accident as well as relaxation exercise using biofeedback to reduce stress for drivers. The following further applications could also be enabled:

1. Vehicle only can be operated when the driver is not feeling too stressed

2. A black box can continuously record all vital signs when driving. In case of an accident all vital signs can be reviewed to see whether the driver had health problems prior to an accident.

While the subject matter has been illustrated in detail in the drawings and foregoing description, such illustration and description are to be considered illustrative or exemplary and not restrictive; the subject matter is not limited to the disclosed embodiments. Other variations to the disclosed embodiments can be understood and effected by those skilled in the art of practicing the claimed subject matter, from a study of the drawings, the disclosure and the appended claims. Use of the verb “comprise” and its conjugates does not exclude the presence of elements other than those stated in a claim or in the description. In the system claims enumerating several units, several of these units can be embodied by one and the same hardware/software item. Use of the indefinite article “a” or “an” preceding an element or step does not exclude the presence of a plurality of such elements or steps. The Figures and description are to be regarded as illustrative only and do not limit the subject matter. Any reference sign in the claims should not be construed as limiting the scope.

1. A method comprising the step of using a plurality of doppler radars disposed on the seat belt or integrated into the seat belt for monitoring vital body signs of a person seated in a seat of a motor vehicle.

2. The method as claimed in claim 1, wherein monitoring the vital body signs of the person seated in the seat of the motor vehicle comprises:
transmitting electromagnetic signals of a certain frequency into the chest of the person seated in the seat of the motor vehicle;
receiving corresponding reflected electromagnetic signals from the chest of the person seated in the seat of the motor vehicle;
processing the corresponding reflected electromagnetic signals to produce output signals representing the rate of change of the doppler signal associated with the reflected electromagnetic signal, the rate of change with respect to time;
comparing the output signals and selecting the best output signal based on a criteria; and
calculating at least one parameter representative of the vital body sign of the person seated based on the selected best output signal.

3. The method as claimed in claim 2, wherein comparing the output signals and selecting the best output signal comprises selecting the best output signal based on heart signal of the person seated in the motor vehicle.

4. The method as claimed in claim 3, wherein selecting the best output signal based on the heart signal further comprises:
extracting characteristic points from the plurality of radar sensors using the time derivatives of all the radar channels, wherein the characteristic points mark transitions between different phases of the heart's pumping cycles;
searching for repeating patterns of the extracted characteristic points; and
selecting the best output signal based on the number of characteristic points in one repeating pattern.

5. The method as claimed in claim 1 wherein the pluralities of doppler radars emit continuous wave electromagnetic signals at a frequency in a range between 400 MHz and 5 GHz.

6. The method as claimed in claim 1, wherein the monitored information about the vital body signs of the person are forwarded to a higher-order system for further processing for at least one of the following purposes:
detecting momentary sleep of the person seated;
classifying the health condition of the person seated; or
giving feedback on the health condition of the person seated.

7. The method as claimed in claim 1, further comprising:
generating an alarm signal when the monitored information about the vital body signs of the person seated indicates a life-threatening or abnormal situation.

8. A device comprising a plurality of doppler radars disposed on the seat belt or integrated into the seat belt to monitor vital body signs of a person seated in a seat of a motor vehicle.

9. A system for monitoring vital body signs of a person seated in a seat of a motor vehicle, the system comprising:
a plurality of transducers and antennas to transmit electromagnetic signals of a certain frequency into the chest of the person and receive corresponding reflected electromagnetic signals from the chest of the person;
a processing unit comprising:
a first processing unit, coupled to the plurality of antennas to process the reflected electromagnetic signals and produce output signals, the output signals representing the rate of change of the doppler signal associated with the reflected signal, the rate of change with respect to time;
a second processing unit, arranged to compare the output signals and select the best output signal based on a criteria; and
a third processing unit, arranged to calculate at least one parameter representative of the vital body sign of the person seated in the seat of the motor vehicle based on the selected best output signal.
